(19) United States
(12) Patent Application Publication
(10) Pub. No.: US 2025/0182319 A1
(43) Pub. Date: Jun. 5, 2025

(54) INFORMATION PROCESSING METHODS AND INFORMATION PROCESSING SYSTEM

(71) Applicant: Panasonic Holdings Corporation, Osaka (JP)

(72) Inventors: NAOMI SATOI, Osaka (JP); HIROTO YANAGAWA, Osaka (JP); HITOSHI TOMINAGA, Kyoto (JP); YUMIKO OHNO, Osaka (JP); AKIKO TAKIMOTO, Osaka (JP); YUKIKO UCHIDA, Kyoto (JP); MASATAKA NAKAYAMA, Kyoto (JP)

(21) Appl. No.: 19/052,371

(22) Filed: Feb. 13, 2025

Related U.S. Application Data
(63) Continuation of application No. PCT/JP2023/027989, filed on Jul. 31, 2023.

(30) Foreign Application Priority Data
Aug. 31, 2022 (JP) 2022-137669

(51) Int. Cl. G06T 7/70; A61B 5/16; G06V 40/20

(52) U.S. Cl. CPC G06T 7/70; A61B 5/163; A61B 5/165; G06V 40/20; G06T 2207/30201

(57) ABSTRACT

An information processing method includes: by using a processor, acquiring at least one index selected from an index group that includes an amount of variation in a face position of a user, an amount of variation in a face orientation of the user, an amount of variation in a gaze direction of the user, and an eye closure percentage of the user, and determining, based on the at least one acquired index, whether or not the user is in a divergent thinking state.

### BACKGROUND

### 1. Technical Field

The present disclosure relates to information processing methods and an information processing system.

### 2. Description of the Related Art

Creative thinking of people includes convergent thinking, in which people logically proceed from known information to arrive at a single solution, and divergent thinking, in which people generate new ideas by thinking through known information.

### SUMMARY

One non-limiting and exemplary embodiment provides information processing methods and the like that enable objective evaluation of examinees' creative thinking states.

In one general aspect, the techniques disclosed here feature an information processing method including: by using a processor, acquiring at least one index selected from an index group that includes an amount of variation in a face position of a user, an amount of variation in a face orientation of the user, an amount of variation in a gaze direction of the user, and an eye closure percentage of the user, and determining, based on the at least one acquired index, whether or not the user is in a divergent thinking state.

This comprehensive or specific aspect may be realized by a system, device, integrated circuit, computer program, or recording medium such as a computer-readable compact-disk read-only memory (CD-ROM), or any combination of a system, device, integrated circuit, computer program, and recording medium.

The information processing methods according to the present disclosure enable objective evaluation of examinees' creative thinking states.

It should be noted that general or specific embodiments may be implemented as a system, a method, an integrated circuit, a computer program, a storage medium, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1A to 1D are diagrams for describing and illustrating, for each thinking state, the amounts of variation in examinees' face positions and other information;

FIG. 2 is a diagram for describing and schematically illustrating an external view of a biometric measurement device and an example of its use state according to an embodiment;

FIG. 3 is a block diagram illustrating an example of the configuration of the biometric measurement device according to the embodiment;

FIG. 4 is a flowchart illustrating a method for determining the thinking state of a user from their biometric measurement data, the method being performed by the biometric measurement device according to the embodiment;

FIG. 5 is a diagram for describing and illustrating an example of thinking states determined by the biometric measurement device according to the embodiment;

FIG. 6A is a diagram for describing a first example of display of a determination result from the biometric measurement device according to the embodiment;

FIG. 6B is a diagram for describing a second example of display of a determination result according to the embodiment;

FIG. 6C is a diagram for describing a third example of display of a determination result according to the embodiment;

FIG. 6D is a diagram for describing a fourth example of display of a determination result according to the embodiment;

FIG. 6E is a diagram for describing a fifth example of display of a determination result according to the embodiment;

FIG. 6F includes diagrams for describing a sixth example of display of determination results according to the embodiment;

FIG. 6G is a diagram for describing a seventh example of display of a determination result according to the embodiment;

FIG. 7 is a flowchart illustrating an example of a process for controlling lighting in accordance with a determination result according to the embodiment;

FIG. 8 is a diagram for describing a relationship between the illuminance and color temperature of lighting and the user's thinking state under lighting control according to the embodiment; and

FIG. 9 is a flowchart illustrating an example of a process regarding the update of threshold information according to the embodiment.

### DETAILED DESCRIPTIONS

### Underlying Knowledge Forming Basis of the Present Disclosure

Before describing the embodiments of the present disclosure, the findings identified by the inventors of the present application will be described.

Against the backdrop of labor shortages in an aging society with a declining birthrate, the government is promoting reforms in the way people work. Companies are working to create various mechanisms to improve the intellectual productivity of their employees. Employee motivation and office space are closely linked, and environmental design or space control is being developed to increase intellectual productivity.

To increase intellectual productivity, it is important not only that employees are able to concentrate on their work, but also that they are able to think more creatively. Creative thinking includes convergent thinking, in which people logically proceed from known information to arrive at a single solution, and divergent thinking, in which people generate new ideas by thinking through known information.

Although there have been studies on methods for evaluating the effects of the surrounding environment on thinking states, standardized, objective, and quantitative indices have not been established.

Specifically, methods for estimating the degree of concentration of a subject on the basis of data obtained from a sensor monitoring the subject have been proposed in Japanese Unexamined Patent Application Publication Nos. 2021-23492, 2016-224142, 2021-35499, and 2020-8278. However, there is no disclosure of a mechanism for objectively evaluating the subject's thinking state in Japanese Unexamined Patent Application Publication Nos. 2021-23492, 2016-224142, 2021-35499, and 2020-8278.

The present disclosure provides information processing methods and the like that enable objective evaluation of people's creative thinking states.

In the following, examples of biometric information obtained when examinees were asked to perform tasks that require convergent thinking and tasks that require divergent thinking.

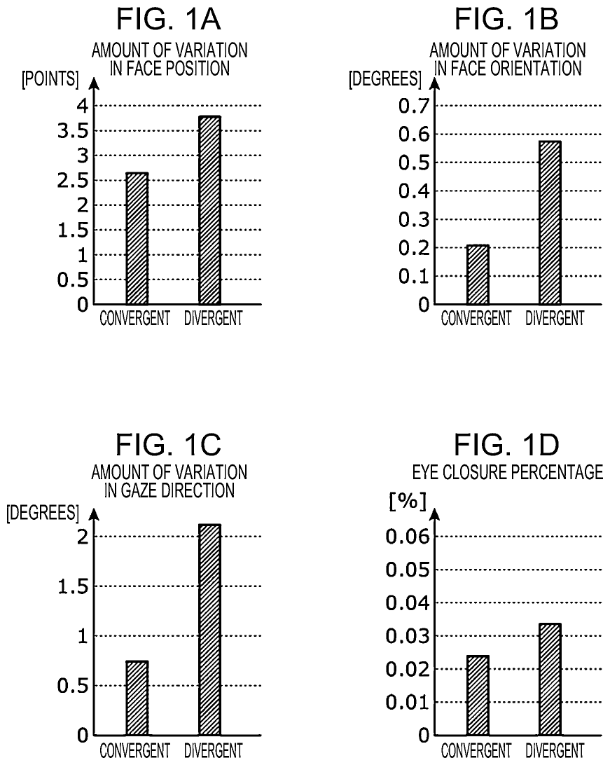

FIGS. 1A to 1D are graphs illustrating examples of biometric information obtained when 50 examinees were asked to perform tasks that require convergent thinking and tasks that require divergent thinking.

The following is a detailed description of the methods used to acquire the biometric information illustrated in FIGS. 1A to 1D.

The Japanese version of the Remote Association Task (RAT) was used as a verbal task, and the Raven Task (Raven) was used as a non-verbal task, both of which require convergent thinking.

The Unusual Uses Test (UUT) was used as a verbal task and the Figural Divergent Task (FDT) was used as a non-verbal task, both of which require divergent thinking.

RAT was developed by Mednick (see S. Mednick.: The associative basis of the creative process. Psychol. Rev. 69, 220-232, (1962)). Mednick presented experiment participants with three words that may seem to have no commonality at first glance (for example, “Pure,” “Blue,” and “Fall”) and asked them to find a common word (“Water”) associated with each word.

RAT has been revised to include tasks created in languages other than English, and the tasks themselves have been revised. In this experiment, we used the questions created by Orita et al. (see R. Orita, M. Hattori, Y. Nishida.: Development of a Japanese Remote Associates Task as insight problems. Japanese J. Psychol. 89, 376-386, (2018)). The instructions for the RAT task given to the examinees were as follows. “Please provide your answer verbally, using a kanji that can be connected to all three kanjis to form words. The answer should be provided verbally, using a word formed with the left kanji. For example, if the three kanjis are “ [symbol] ”, then “ [symbol] ” is a correct answer. This is because the words would be “ [symbol] , [symbol] , [symbol] ”. In this case, you need to answer“ [symbol] ”. Please answer as quickly and accurately as possible. As soon as you answer, we will move on to the next question. If 45 seconds elapse without an answer, we will also move on to the next question. Correct answers will be scored 1 point, and incorrect answers and timeouts will be scored −1 point”.

Raven's Progressive Matrices is widely used as a test to assess intelligence and reasoning ability (see & R. Raven, J. C., Court, J. H.: Raven's progressive matrices.; Oxford Psychologists Press, 1998). Matzen et al. analyzed the types of relationships that appear in the Standard Progressive Matrices (SPM) of Raven's Progressive Matrices and created a software tool that can generate a very large number of matrices by combining the same types of relationships in accordance with parameters chosen by the experimenter (see L. E. Matzen, Z. O. Benz, K. R. Dixon, J. Posey, J. K. K roger, A. E. Speed.: Recreating Raven's: Software for systematically generating large numbers of Raven-like matrix problems with normed properties. Behav. Res. Methods 42, 525-541, (2010)). In this experiment, we used that publicly available software to create Raven tasks. The instructions for a Raven task given to the examinees were as follows. “Here, there is a series of geometric figures that follows a certain rule. Please select one out of the eight options for the figure that goes in the missing area and provide your answer verbally. The answer should be provided verbally, with a number out of 1 to 8 attached to the respective options. In this case, “1” is the correct answer. Please answer as quickly and accurately as possible. As soon as you answer, we will move on to the next question. If 45 seconds elapse without an answer, we will also move on to the next question. Correct answers will be scored 1 point, and incorrect answers and timeouts will be scored −1 point ”.

UUT is a task in which examinees name many unique uses for objects, and the degree of divergent thinking is measured. The instructions for the UUT task given to the examinees were as follows. “Within three minutes, say out loud as many unusual uses for “an object” as possible. There are no correct answers, so you may start with the first one that comes to mind and say it out loud. Please say your answer clearly and specifically. For example, “an object” is “a sock”. In this case, “roll up the sock and use it as a beanbag” is an unusual use. “Wear the sock” is normal usage. Scoring will be based on the number and originality of answers. Please provide as many answers as you can with as much originality as possible. There are no incorrect answers, and no point deductions.” For the measurement of biometric information described in FIGS. 1A to 1D, the examinees were asked to provide as many unusual uses of “a ping pong ball” as they can within three minutes.

FDT is a task in which examinees provide as many possible interpretations of figures as they can. (see M. A. Wallach, Nathan Kogan.: Modes of thinking in young children; A study of the creativity-intelligence distinction; Holt, Rinehart and Winston, 1965). The instructions for the FDT task given to the examinees were as follows. “Within three minutes, say out loud as many possible interpretations of a figure as you can. There are no correct answers, so you may start with the first one that comes to mind and say it out loud. Please provide your answer clearly and specifically. For example, the figure is “□” (namely, a white square figure). In this case, a possible interpretation of the figure is “bread”. Scoring will be based on the number and originality of answers. Please provide as many answers as you can with as much originality as possible. There are no incorrect answers, and no point deductions.” In this experiment, the examinees were asked to provide as many possible interpretations of “+” (in other words, a figure formed by a horizontal line segment and a line segment orthogonal to the line segment) as they can within three minutes.

For each examinee, regarding a one-minute period in which the examinee was not performing a task (also called the resting state) and also a three-minute period in which the examinee was performing a task (also called the execution state), the mean and standard deviation of the examinee's face position, those of the examinee's face orientation, and those of the examinee's gaze direction were calculated. Regarding the one-minute period in the resting state, the examinee's eye closure percentage was also calculated, and the mean value of the eye closure percentages over the three-minute period in the execution state was calculated. The eye closure percentage was defined as the percentage of time the eyes were closed per minute. The three-minute period in the execution state was divided into one-minute segments to calculate three values as the eye closure percentage in the execution state. The mean value of these three values was treated as the eye closure percentage in the execution state.

Next, for each examinee, the difference between the standard deviation in the execution state and the standard deviation in the resting state was calculated for each of the examinee's face position, face orientation, and gaze direction. Standard deviation is referred to as the amount of variation as a value representing the degree of variation in data and can be used to compare the amount of variation in the value in the resting state with the amount of variation in the value in the execution state. Moreover, for each examinee, the difference between the mean value of the eye closure percentage in the execution state and the mean value of the eye closure percentage in the resting state was calculated. Lastly, the mean value of the above differences of all examinees' face positions, the mean value of the above differences of all examinees' face orientations, the mean value of the above differences of all examinees' gaze directions, and the mean value of the above differences of all examinees' eye closure percentages obtained during the task requiring convergent thinking were compared with those obtained during the task requiring divergent thinking.

In addition to the above standard deviation of the examinee's face position and so on, an index (also called a variation index) indicating variations in the examinee's face position and so on as amounts of variation can also be used. The variation index is, for example, variance.

As the amounts of variation, the differences between the variation indices of the examinee's face position and so on in the resting state and the variation indices of the examinee's face position and so on in the execution state can also be used. Specifically, for example, the values obtained by subtracting the variation indices of the examinee's face position and so on in the resting state from the variation indices of the examinee's measured face position and so on can be used as the amounts of variation. The variation indices of the examinee's face position and so on in the resting state may be measured in advance and stored in a memory device.

FIG. 1A illustrates, for each of the convergent thinking state (abbreviated as “convergent” in the drawing) and the divergent thinking state (abbreviated as “divergent” in the drawing), the mean value of the differences between the amounts of variation in face position for all examinees in the execution state and the amounts of variation in face position for all examinees in the resting state. The amounts of variation in face position for all examinees indicate how much their faces moved from reference positions in captured images of the examinees' faces. In this case, as the amounts of variation in face position, normalized values for the amounts of variation in face position are used. The unit of measurement is “points”. Note that as the amounts of variation in face position, values normalized using personal feature values, such as the widths of their faces, may be used, the numbers of pixels on the acquired images may be used, or the lengths calculated from the specifications of the camera and lens (unit: millimeters) may be used.

FIG. 1B illustrates, for each of the convergent thinking state and the divergent thinking state, the mean value of the differences between the amounts of variation in face orientation for all examinees in the execution state and the amounts of variation in face orientation for all examinees in the resting state. The amounts of variation in face orientation for all examinees are the amounts of variation in the three-dimensional orientations of their faces obtained by analyzing captured images of their faces, and indicate how much their face orientations are tilted from reference directions. In this case, as the amounts of variation in face orientation, the angles of their face orientations relative to the reference directions are used.

Compared to the resting state, it is clear that the amounts of variation in face position and face orientation for the examinees are greater in the execution state (namely, in the convergent thinking state or divergent thinking state).

It is generally believed that when people are concentrating on a simple task, the amounts of variation in face position and face orientation are reduced compared to when they are at rest, because they gaze at the position where the task is displayed and unnecessary movements are suppressed. In contrast, it can be seen that when examinees are engaged in creative thinking corresponding to convergent thinking and divergent thinking, the examinees tend to be different from when they are concentrating on a simple task.

Furthermore, it can be seen that the amounts of variation in face position and face orientation are greater when the examinees are thinking divergently than when the examinees are thinking convergently. Thus, when the amounts of variation in face position and face orientation for examinees increase from the resting state, it can be inferred that the examinees are thinking creatively, and when the amounts of variation in face position and face orientation for examinees increase further, it can be inferred that the examinees are moving toward divergent thinking or that the degrees of the divergent thinking are increasing.

FIG. 1C illustrates, for each of the convergent thinking state and the divergent thinking state, the mean value of the differences between the amounts of variation in gaze direction for all examinees in the execution state and the amounts of variation in gaze direction for all examinees in the resting state. The amounts of variation in gaze direction for all examinees are obtained by analyzing captured images of their faces, and indicate how much their gaze directions changed from reference directions. In this case, as the amounts of variation in gaze direction, the angles of the gaze directions relative to the reference directions are used.

FIG. 1D illustrates, for each of the convergent thinking state and the divergent thinking state, the mean value of the above differences between the eye closure percentages for all examinees in the execution state and the eye closure percentages for all examinees in the resting state. Eye closure percentage is the percentage of time that the eyes are closed during a unit period, such as one minute.

Compared to the resting state, it is clear that the amounts of variation in gaze direction and the eye closure percentages are greater in the execution state (namely, in the convergent thinking state or divergent thinking state). This is also the opposite tendency of the general state of people when they are concentrating on a simple task, and this can be considered a characteristic of creative thinking. Furthermore, the amounts of variation in gaze direction and the eye closure percentages are greater when people are thinking divergently than when people are thinking convergently. When the amounts of variation in gaze direction or the eye closure percentages are greater than those in the resting state, it can be inferred that people are thinking creatively and that they are moving toward divergent thinking or the degrees of the divergent thinking are increasing.

For example, in a case where the amount of variation in face orientation and the eye closure percentage increase compared with those in the resting state, it can be inferred that an examinee is moving toward divergent thinking. For example, if only the eye closure percentage increases out of the amount of variation in face orientation and the eye closure percentage, the examinee may be feeling drowsy rather than moving toward the divergent thinking state. Thus, combining at least one piece of biometric information of the amount of variation in face position, the amount of variation in face orientation, or the amount of variation in gaze direction may also improve the accuracy of thinking state estimation.

In the following, examples of techniques that can be obtained from the content disclosed in the present specification are illustrated, and the effects, for example, obtained from such techniques are described.
(1) An information processing method including: by using a processor, acquiring at least one index selected from an index group that includes an amount of variation in a face position of a user, an amount of variation in a face orientation of the user, an amount of variation in a gaze direction of the user, and an eye closure percentage of the user, and determining, based on the at least one acquired index, whether or not the user is in a divergent thinking state.

According to the above aspect, whether or not the user (examinee) is in the divergent thinking state can be determined using the at least one index selected from the index group that includes the amount of variation in the face position of the user, the amount of variation in the face orientation of the user, the amount of variation in the gaze direction of the user, and the eye closure percentage of the user. There is no known technique to determine whether or not a user is in the divergent thinking state from the amount of variation in the face position of the user, the amount of variation in the face orientation of the user, the amount of variation in the gaze direction of the user, and the eye closure percentage of the user. According to the above information processing method, it is possible to determine with higher accuracy whether or not a user is in the divergent thinking state from the amount of variation in the face position of the user, the amount of variation in the face orientation of the user, the amount of variation in the gaze direction of the user, and the eye closure percentage of the user. In this manner, the above information processing method enables objective evaluation of an examinee's creative thinking state.
(2) The information processing method according to (1), in which the determining includes comparing the at least one index with a first threshold corresponding to the at least one index, and determining, in a case where the at least one index is determined to be greater than or equal to the first threshold, that the user is in the divergent thinking state.

According to the above aspect, whether or not the user is in the divergent thinking state can be easily determined by using the magnitude determination between the index and the first threshold. In this manner, the above information processing method enables easier objective evaluation of an examinee's creative thinking state.
(3) The information processing method according to (2), in which the determining includes comparing the at least one index with a second threshold corresponding to the at least one index and different from the first threshold, and determining, in a case where the at least one index is determined to be less than the first threshold and greater than or equal to the second threshold, that the user is in a convergent thinking state.

According to the above aspect, whether or not the user is in the convergent thinking state can be easily determined by using the magnitude determination between the index and the first and second thresholds. In this manner, the above information processing method enables easier objective evaluation of an examinee's creative thinking state.
(4) The information processing method according to (2) or (3), in which the determining includes determining, for each of indices selected from the index group, whether or not the index is greater than or equal to the first threshold corresponding thereto.

According to the above aspect, since indices are used as the at least one index selected from the index group to determine whether or not the user is in the divergent thinking state, the validity of the determination is improved to contribute to improving the accuracy of the determination result. In this manner, the above information processing method enables objective and highly accurate evaluation of an examinee's creative thinking state.
(5) The information processing method according to (4), in which the determining includes determining, in a case where the amount of variation in the face orientation of the user is determined to be greater than or equal to the first threshold corresponding thereto and where the amount of variation in the gaze direction of the user is determined to be greater than or equal to the first threshold corresponding thereto, that the user is in the divergent thinking state.

According to the above aspect, whether or not the user is in the divergent thinking state is determined using, as the at least one index selected from the index group, the amount of variation in face orientation and the amount of variation in gaze direction. The inventors of the present application have confirmed that for each of the amount of variation in face orientation and the amount of variation in gaze direction, the difference between the resting state and the divergent thinking state is greater than that for the other indices. Thus, using the amount of variation in face orientation and the amount of variation in gaze direction as the at least one index improves the validity of the determination, and this contributes to improving the accuracy of the determination result. In this manner, the above information processing method enables objective and highly accurate evaluation of an examinee's creative thinking state.
(6) The information processing method according to (4), in which the determining includes determining a degree of the divergent thinking state of the user in a unit period, based on a count of indices that are greater than or equal to the first thresholds corresponding thereto among the indices acquired in the unit period.

According to the above aspect, whether or not the user is in the divergent thinking state is determined and also the degree of the divergent thinking state is determined using the indices as the at least one index selected from the index group. This enables to evaluate an examinee's creative thinking state in terms of the degree of the divergent thinking state. Thus, the above information processing method enables objective and highly accurate evaluation of an examinee's creative thinking state.
(7) The information processing method according to (3), in which the determining includes determining, using the at least one index acquired in each of unit periods, whether or not the user is in the divergent thinking state and whether or not the user is in the convergent thinking state in each of the unit periods, generating a graph representing a percentage of a period during which the user is determined to be in the convergent thinking state and a percentage of a period during which the user is determined to be in the divergent thinking state, and displaying the graph.

According to the above aspect, since the graph representing the percentage of the thinking state of the user in each of the periods is displayed, it contributes to allowing the viewer of the graph to grasp the thinking state of the user over the periods at a glance. Thus, the above information processing method enables objective evaluation of an examinee's creative thinking state over periods and furthermore allows the viewer to grasp the thinking state easily.
(8) The information processing method according to any one of (2) to (7), further including: acquiring identification information for identifying the user, and acquiring threshold information corresponding to the identification information. The first threshold is determined based on the threshold information.

According to the above aspect, since the first threshold used to perform a magnitude determination on the index is determined on the basis of the threshold information, the first threshold can be made more appropriate, and it can be appropriately determined whether or not the thinking state of the user is the divergent thinking state. Thus, the above information processing method enables objective and highly accurate evaluation of an examinee's creative thinking state.
(9) The information processing method according to (8), further including: acquiring evaluation information obtained by evaluating a thinking state of the user using a method different from the determination process, and updating the threshold information based on a determination result of a determined thinking state of the user and the evaluation information.

According to the above aspect, since the first threshold is updated on the basis of the subjective evaluation information obtained by evaluating the state related to the user's thinking using a method different from the above determination process, this contributes to bringing the determination result of the subsequent state of the user closer to the evaluation performed by the user. Thus, the above information processing method enables objective evaluation of an examinee's creative thinking state while bringing the determination result of the examinee's thinking state closer to the examinee's subjective evaluation.
(10) The information processing method according to (3), further including: controlling, in a case where the at least one index is determined to be less than the first threshold and greater than or equal to the second threshold, a lighting device, which illuminates a surrounding area of the user, to lower a color temperature of illuminating light and increase illuminance of the illuminating light.

According to the above aspect, in a case where the state related to the user's thinking is determined to be the convergent thinking state, it is possible to appropriately induce the user to the divergent thinking state by using the lighting device. Thus, according to the above information processing method, the examinee's creative thinking state can be objectively evaluated, and the user determined to be in the convergent thinking state can be induced to the divergent thinking state.
(11) The information processing method according to any one of (2) to (9), further including: controlling, in a case where the at least one index is determined to be greater than or equal to the first threshold, a lighting device, which illuminates a surrounding area of the user, to increase a color temperature of illuminating light and increase illuminance of the illuminating light.

According to the above aspect, in a case where the state related to the user's thinking is determined to be the divergent thinking state, it is possible to appropriately induce the user to the convergent thinking state by using the lighting device. Thus, according to the above information processing method, an examinee's creative thinking state can be objectively evaluated, and the user determined to be in the divergent thinking state can be induced to the convergent thinking state.
(12) The information processing method according to (3), further including: controlling, in a case where the at least one index is determined to be less than the second threshold, a lighting device, which illuminates a surrounding area of the user, to (i) lower a color temperature of illuminating light and increase illuminance of the illuminating light, or (ii) increase a color temperature of illuminating light and increase illuminance of the illuminating light.

According to the above aspect, in a case where the state related to the user's thinking is determined to be neither the divergent thinking state nor the convergent thinking state, it is possible to appropriately induce the user to the divergent thinking state or the convergent thinking state by using the lighting device. Thus, according to the above information processing method, an examinee's creative thinking state can be objectively evaluated, and the user determined to be in neither the divergent thinking state nor the convergent thinking state can be induced to the divergent thinking state or the convergent thinking state.
(13) The information processing method according to any one of (1) to (12), in which the acquiring includes acquiring an image generated by an image capturing device that captures the user, and acquiring the at least one index by performing an analysis process on the acquired image.

According to the above aspect, since the indices are acquired by performing the analysis process on the image in which the user appears, the process for acquiring indices can be easily realized. Thus, the above information processing method enables easier objective evaluation of an examinee's creative thinking state.
(14) An information processing method including: by using a processor, acquiring target information indicating a target state that is a state targeted by a user, acquiring at least one index selected from an index group that includes an amount of variation in a face position of the user, an amount of variation in a face orientation of the user, an amount of variation in a gaze direction of the user, and an eye closure percentage of the user, determining a thinking state of the user based on the at least one index, comparing the target state indicated by the target information with the determined thinking state of the user, and performing, in a case where the target state and the determined thinking state of the user are different as a result of the comparison, control to change a characteristic of illuminating light emitted from a lighting device, which illuminates a surrounding area of the user.

According to the above aspect, in a case where the thinking state of the user determined from, for example, the amount of variation in the face position of the user is different from the target state, the thinking state of the user can be appropriately guided to the target state by using the lighting device. Thus, the above information processing method enables objective evaluation of a creative thinking state of an examinee and also allows the thinking state of the user to be guided to the target state.
(15) An information processing system including: an image capturing device that captures a user to generate an image, a processing circuit, a memory device, and a display device. The processing circuit acquires at least one index selected from an index group that includes an amount of variation in a face position of the user, an amount of variation in a face orientation of the user, an amount of variation in a gaze direction of the user, and an eye closure percentage of the user in a unit period by performing an analysis process on the image generated by the image capturing device, acquires, from the memory device, threshold information indicating a first threshold corresponding to the at least one index, performs a determination process for determining whether or not the user is in a divergent thinking state in the unit period, based on a determining result as to whether or not the at least one index is greater than or equal to the first threshold in the unit period, and causes the display device to display an image corresponding to a result of the determination process.

According to the above aspect, substantially the same effects as the above information processing methods are achieved.

These comprehensive or specific aspects may be realized by a system, device, integrated circuit, computer program, or recording medium such as a computer-readable CD-ROM, or any combination of a system, device, integrated circuit, computer program, or recording medium.

In the following, embodiments will be specifically described with reference to the drawings.

Note that the embodiments described below are all examples, each of which is either comprehensive or specific. For example, the numerical values, shapes, materials, structural elements, structural-element arrangement positions and connection forms, steps, and sequences of steps illustrated in the following embodiments are examples and are not intended to limit the present disclosure. Among the structural elements in the following embodiments, the structural elements that are not described in the independent claims that indicate the highest level of concept are described as optional structural elements.

### Embodiments

In the present embodiments, information processing methods and the like that enable objective evaluation of an examinee's creative thinking state will be described.

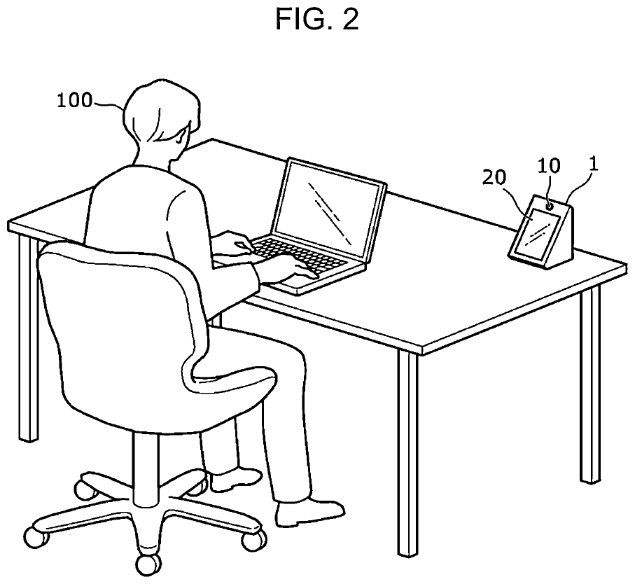

FIG. 2 is a diagram for describing and schematically illustrating an external view of a biometric measurement device 1 and an example of its use state according to the present embodiment.

The biometric measurement device 1 is an information processing device that measures the biometric information of a user 100.

The biometric measurement device 1 includes a camera 10 and a display device 20. The camera 10 is placed away from the user 100 so as to capture the head (more specifically, the face) of the user 100, who is an examinee, when the biometric measurement device 1 measures their biometric information. While the user 100 is engaging in a task, the camera 10 captures the face of the user 100 and acquires an image information signal in which the face of the user 100 is captured. The camera 10 may repeatedly capture the face of the user 100. The image signal may be acquired in real time or every time image capturing is performed.

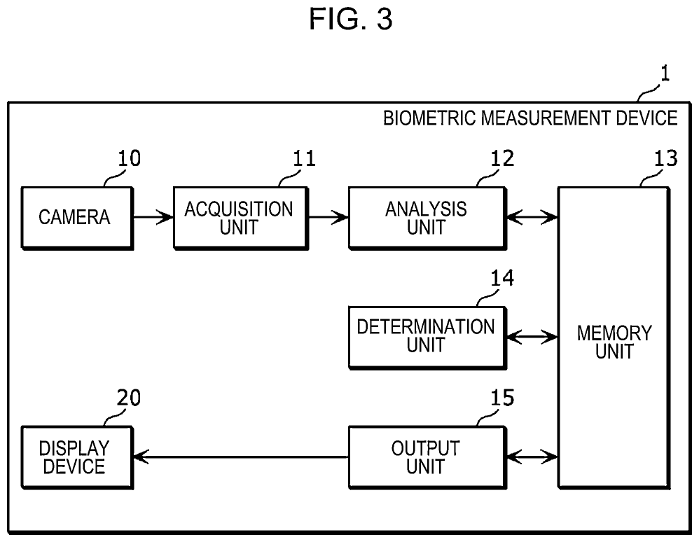

FIG. 3 is a block diagram illustrating an example of the configuration of the biometric measurement device 1 according to the present embodiment.

As illustrated in FIG. 3, the biometric measurement device 1 includes the camera 10, an acquisition unit 11, an analysis unit 12, a memory unit 13, a determination unit 14, an output unit 15, and the display device 20. The analysis unit 12, the memory unit 13, the determination unit 14, and the output unit 15 can be realized by a processor of the biometric measurement device 1 executing a predetermined program.

The acquisition unit 11 converts image information signals acquired by the camera 10 into images and acquires the images.

The analysis unit 12 applies an analysis process (for example, face recognition processing) to images acquired by the acquisition unit 11, in which the face of the user 100 appears, to extract feature points such as the outline of the face, eyes, or mouth of the user 100. The analysis unit 12 uses the extracted feature points to identify at least one of the face position, the face orientation, the gaze direction, or the eye closure percentage of the user 100. The analysis unit 12 calculates at least one index selected from the amount of variation in the face position of the user 100, the amount of variation in the face orientation of the user 100, the amount of variation in the gaze direction of the user 100, and the eye closure percentage of the user 100 and stores the calculated index into the memory unit 13. The above index is also referred to as biometric information. The amount of variation in face position, the amount of variation in face orientation, the amount of variation in gaze direction, and the eye closure percentage are also referred to as an index group. The analysis unit 12 acquires at least one index selected from the above index group in the above-described manner.

The amount of variation in the face position of the user 100 corresponding to a time t0 and a time t1 may be determined on the basis of the face position of the user 100 being in a resting state at the time t0 and the face position of the user 100 obtained at the time t1.

The amount of variation in the face orientation of the user 100 corresponding to the time t0 and the time t1 may be determined on the basis of the face orientation of the user 100 being in the resting state at the time to and the face orientation of the user 100 obtained at the time t1.

The amount of variation in the gaze direction of the user 100 corresponding to the time t0 and the time t11 may be determined on the basis of the gaze direction of the user 100 being in the resting state at the time t and the gaze direction of the user 100 obtained at the time t1. Indices may be acquired on the basis of a moving image acquired by a single camera, and a thinking state may be determined on the basis of these indices. This enables simpler and more accurate determination of a thinking state, compared with complex configurations using devices.

On the basis of a moving image acquired by a single camera, the amount of variation in face orientation and the amount of variation in gaze direction may be derived, but the face position and the eye closure percentage do not have to be derived. This allows a highly accurate determination to be made using the amount of variation in face orientation and the amount of variation in gaze direction, while omitting the process of deriving other indices, thereby making it possible to reduce the processing load.

The memory unit 13 is a memory device that stores the indices calculated by the analysis unit 12. Moreover, the memory unit 13 prestores thresholds (specifically, first thresholds and second thresholds) corresponding to the indices of the amount of variation in face position, amount of variation in face orientation, amount of variation in gaze direction, and eye closure percentage. The thresholds corresponding to the indices are used for comparison with the indices and are the basis for determining whether or not the user 100 is in a divergent thinking state. The indices and thresholds stored in the memory unit 13 are read out by the determination unit 14. The processor may cause the memory unit 13 not to store information that is not used in an estimation process (for example, any of the face position, the face orientation, the gaze direction, and the eye closure percentage). This makes it possible to reduce unnecessary consumption of the memory unit 13.

The determination unit 14 determines the thinking state of the user 100 on the basis of the indices recorded in the memory unit 13, namely at least one index selected from among the amount of variation in face position, the amount of variation in face orientation, the amount of variation in gaze direction, and the eye closure percentage. The determination of the thinking state includes at least a determination as to whether or not the user 100 is in the divergent thinking state. The determination unit 14 provides the determination result to the output unit 15.

For example, the above determination includes comparing the at least one index described above with the first threshold corresponding to the at least one index described above. In a case where the at least one index described above is determined to be greater than or equal to the above first threshold in the above determination, the determination unit 14 determines that the user 100 is in the divergent thinking state.

The above determination may include comparing the at least one index described above with the second threshold corresponding to the at least one index described above and different from the above first threshold, and determining, in a case where the at least one index described above is less than the above first threshold and greater than or equal to the above second threshold, that the user 100 is in a convergent thinking state.

Note that the at least one index described above may be indices. That is, the above determination may include determining as to whether each of the indices selected from the above index group is greater than or equal to the first threshold corresponding to the index.

More specifically, the at least one index described above may be the amount of variation in face orientation and the amount of variation in gaze direction. That is, in a case where it is determined that the amount of variation in face orientation is greater than or equal to the first threshold corresponding thereto and where the amount of variation in gaze direction is greater than or equal to the first threshold corresponding thereto, the above determination may include determining that the user 100 is in the divergent thinking state.

The output unit 15 acquires a determination result from the determination unit 14 and converts the acquired determination result into a format that can be output. For example, in a case where the display device 20 outputs the determination result, the output unit 15 generates an image indicating the determination result and provides the image to the display device 20.

The display device 20 displays an image indicating a determination result from the determination unit 14.

Note that the biometric measurement device 1 may have other output devices (for example, speakers or communication interfaces) instead of or together with the display device 20. In a case where the biometric measurement device 1 has a speaker, the output unit 15 generates audio data indicating the determination result, provides the audio data to the speaker, and causes the speaker to output audio. In a case where the biometric measurement device 1 has a communication interface, the output unit 15 generates communication data including information indicating the determination result and provides the communication data to the communication interface and to other communication devices via the communication interface.

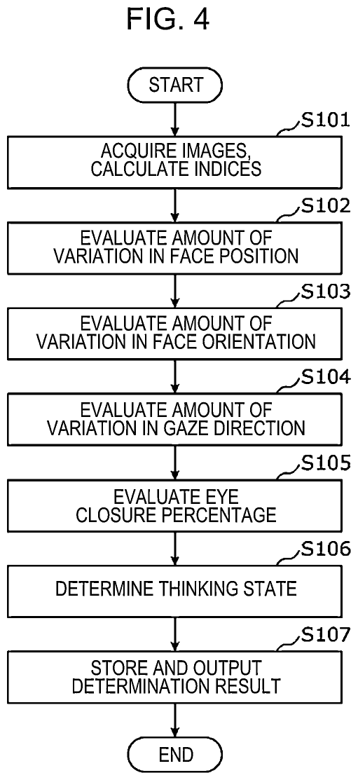

FIG. 4 is a flowchart illustrating a method for determining the thinking state of the user 100 from their biometric measurement data, the method being performed by the biometric measurement device 1 according to the present embodiment.

In Step S101, the acquisition unit 11 uses the camera 10 to acquire an image in which the face of the user 100 is captured. The analysis unit 12 calculates at least one index from images acquired by the acquisition unit 11 and stores the at least one index into the memory unit 13. In the following, as an example, a case will be described in which four indices such as face position, face orientation, gaze direction, and eye closure percentage are calculated as the at least one index.

The analysis unit 12 may calculate the at least one index after reducing the resolution of the images. This can reduce the amount of processing of the processor.

In Step S102, the determination unit 14 reads out the face positions stored in the memory unit 13 and calculates the amount of variation in face position. Moreover, the determination unit 14 evaluates the calculated amount of variation in face position. More specifically, the determination unit 14 determines whether or not the calculated amount of variation in face position is greater than or equal to the first threshold. In a case where the above amount of variation is determined to be less than the first threshold, the determination unit 14 determines whether or not the above amount of variation is greater than or equal to the second threshold.

In Step S103, the determination unit 14 reads out the face orientations stored in the memory unit 13 and calculates the amount of variation in face orientation. Moreover, the determination unit 14 evaluates the calculated amount of variation in face orientation. More specifically, the determination unit 14 determines whether or not the calculated amount of variation in face orientation is greater than or equal to the first threshold. In a case where the above amount of variation is determined to be less than the first threshold, the determination unit 14 determines whether or not the above amount of variation is greater than or equal to the second threshold.

In Step S104, the determination unit 14 reads out the gaze directions stored in the memory unit 13 and calculates the amount of variation in gaze direction. Moreover, the determination unit 14 evaluates the calculated amount of variation in gaze direction. More specifically, the determination unit 14 determines whether or not the calculated amount of variation in gaze direction is greater than or equal to the first threshold. In a case where the above amount of variation is determined to be less than the first threshold, the determination unit 14 determines whether or not the above amount of variation is greater than or equal to the second threshold.

In Step S105, the determination unit 14 reads out the eye closure percentage stored in the memory unit 13 and calculates the eye closure percentage. Moreover, the determination unit 14 evaluates the calculated eye closure percentage. More specifically, the determination unit 14 determines whether or not the calculated eye closure percentage is greater than or equal to the first threshold. In a case where the calculated eye closure percentage is determined to be less than the first threshold, the determination unit 14 determines whether or not the calculated eye closure percentage is greater than or equal to the second threshold.

In Step S106, the determination unit 14 uses the evaluation results obtained in Steps S102 to S105 to determine the thinking state of the user 100. Specifically, in a case where in at least one of the determinations obtained in Steps S102 to S105, the index or indices are greater than or equal to the first threshold or the first thresholds, the determination unit 14 determines that the user 100 is in the divergent thinking state. In a case where the indices in all of the determinations obtained in Steps S102 to S105 are less than the first thresholds and where at least one of the indices is greater than or equal to the second threshold, the determination unit 14 determines that the user 100 is in the convergent thinking state.

In Step S107, the determination unit 14 stores the determination result obtained in Step S106 into the memory unit 13. The output unit 15 outputs the determination result obtained in Step S106 through the display device 20.

It is sufficient that at least one out of Steps S102 to S105 described above be executed. If there is an unexecuted step among Steps S102 to S105, “Steps S102 to S105” in Step S106 is to be read as “steps other than unexecuted steps among Steps S102 to S105”.

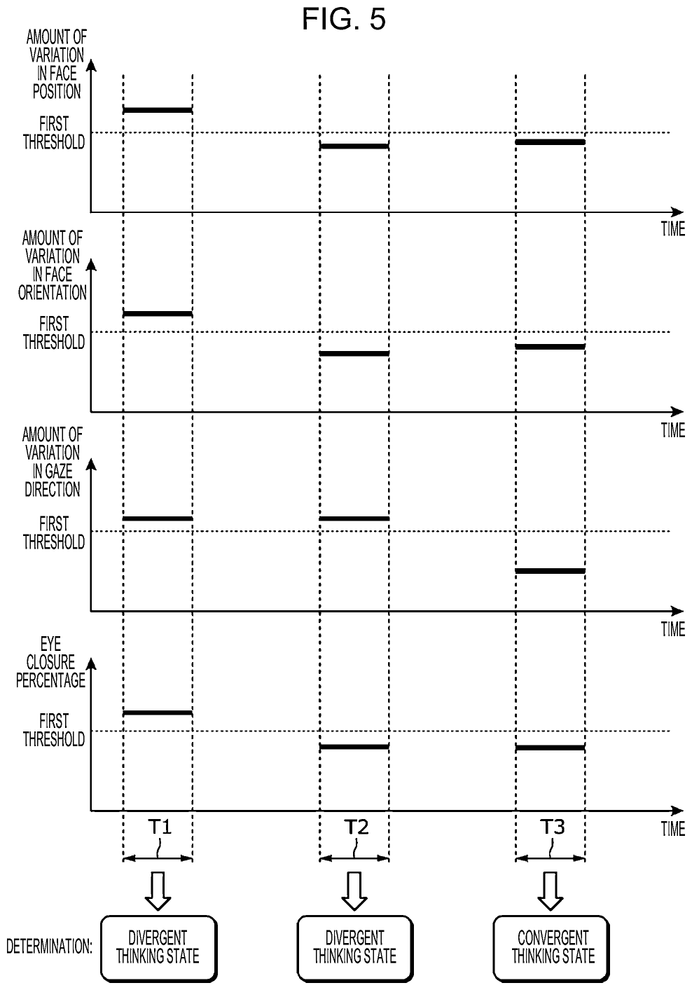

FIG. 5 is a diagram for describing and illustrating an example of thinking states determined by the biometric measurement device 1 according to the present embodiment.

FIG. 5 is used to describe a specific example of a determination performed by the determination unit 14 and indicated by the flowchart in FIG. 4. In the following, time segments T1, T2, and T3 illustrated in FIG. 5 will be described. Note that in the description of FIG. 5, suppose that the indices to be determined in Steps S102 to S105 are all greater than or equal to the second thresholds.

In the case of the time segment T1, the determination unit 14 determines in Step S102 that the amount of variation in face position is greater than or equal to the first threshold, determines in Step S103 that the amount of variation in face orientation is greater than or equal to the first threshold, determines in Step S104 that the amount of variation in gaze direction is greater than or equal to the first threshold, and determines in Step S105 that the eye closure percentage is greater than or equal to the first threshold. On the basis of the determination results obtained in Steps S102 to S105, namely at least one index out of the determinations performed in Steps S102 to S105 being greater than or equal to the first threshold, the determination unit 14 then determines in Step S106 that the thinking state of the user 100 is the divergent thinking state.

In the case of the time segment T2, the determination unit 14 determines in Step S102 that the amount of variation in face position is less than the first threshold, determines in Step S103 that the amount of variation in face orientation is less than the first threshold, determines in Step S104 that the amount of variation in gaze direction is greater than or equal to the first threshold, and determines in Step S105 that the eye closure percentage is less than the first threshold. On the basis of the determination results obtained in Steps S102 to S105, namely at least one index out of the determinations made in Steps S102 to S105 being greater than or equal to the first threshold, the determination unit 14 then determines in Step S106 that the thinking state of the user 100 is the divergent thinking state, and records the result in Step S107.

In this manner, in a case where at least one or more indices are determined to be greater than or equal to the first thresholds, the biometric measurement device 1 determines that the thinking state of the user 100 is the divergent thinking state.

When comparing the time segment T1 and the time segment T2, it can be said that the degree of the divergent thinking state of the user 100 is higher in the time segment T1 in which more indices are determined to be greater than or equal to the first thresholds. The determination unit 14 may thus determine the degree of the divergent thinking state from the count of indices determined to be greater than or equal to the first thresholds and store the determination result into the memory unit 13. The output unit 15 may also output the stored degree of the divergent thinking state.

Note that, together with or instead of the above, the determination unit 14 can evaluate that the greater the difference between the index and the first threshold, the greater the degree of the divergent thinking state. Moreover, the determination unit 14 can evaluate that the longer the time determined to be in the divergent thinking state, the greater the degree of the divergent thinking state.

In the case of the time segment T3, the determination unit 14 determines in Step S102 that the amount of variation in face position is less than the first threshold, determines in Step S103 that the amount of variation in face orientation is less than the first threshold, determines in Step S104 that the amount of variation in gaze direction is less than the first threshold, and determines in Step S105 that the eye closure percentage is less than the first threshold. On the basis of the determination results obtained in Steps S102 to S105, namely the indices in all of the determinations made in Steps S102 to S105 being less than the first thresholds, the determination unit 14 determines in Step S106 that the thinking state of the user 100 is the convergent thinking state.

In this manner, in a case where all of the indices are determined to be less than the first thresholds, the biometric measurement device 1 determines that the thinking state of the user 100 is the convergent thinking state.

In the following, the display of information regarding the state of the user 100 will be described. The information to be presented includes the determination result stored in the memory unit 13 in Step S107 of FIG. 4 and is presented to the user 100 using the display device 20.

The display device 20 presents the thinking state of the user 100 determined by the biometric measurement device 1, and in other words, visualizes the thinking state of the user 100 (so-called visualization).

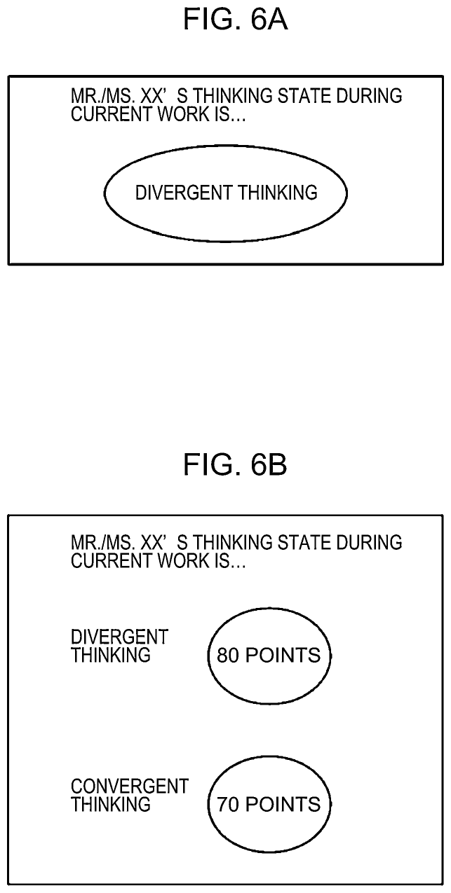

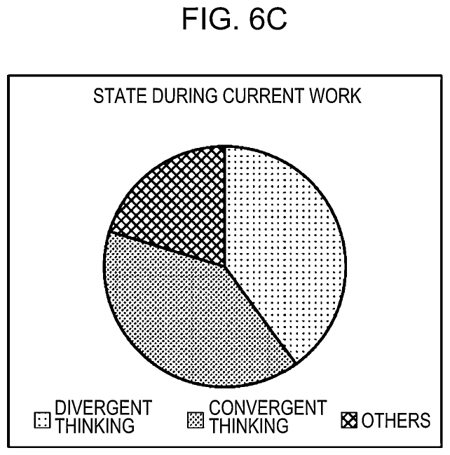

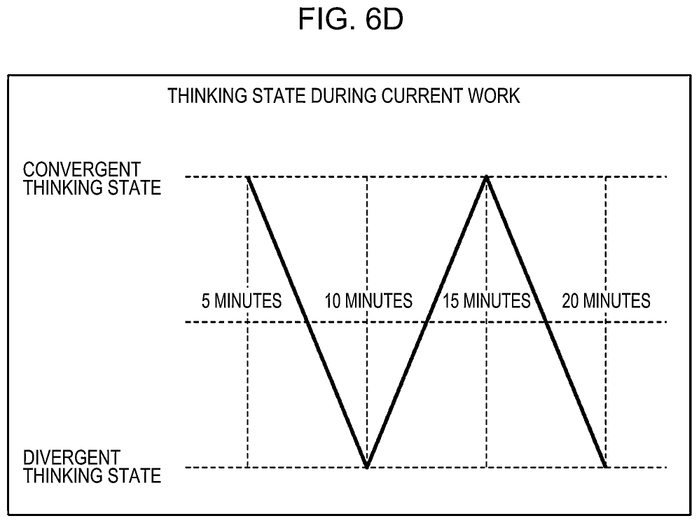

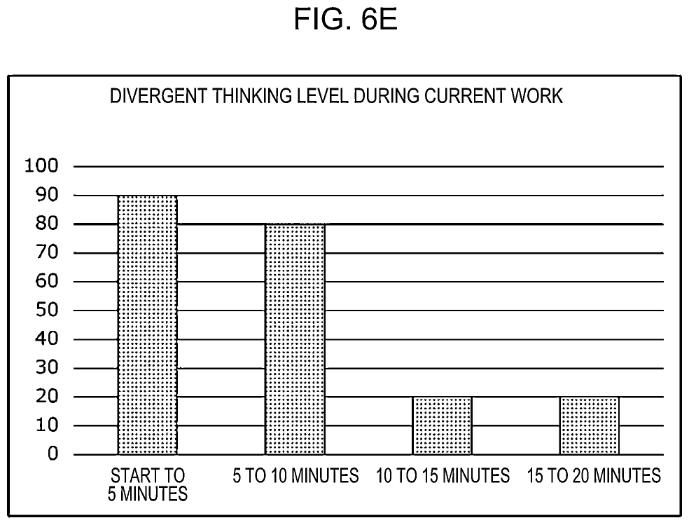

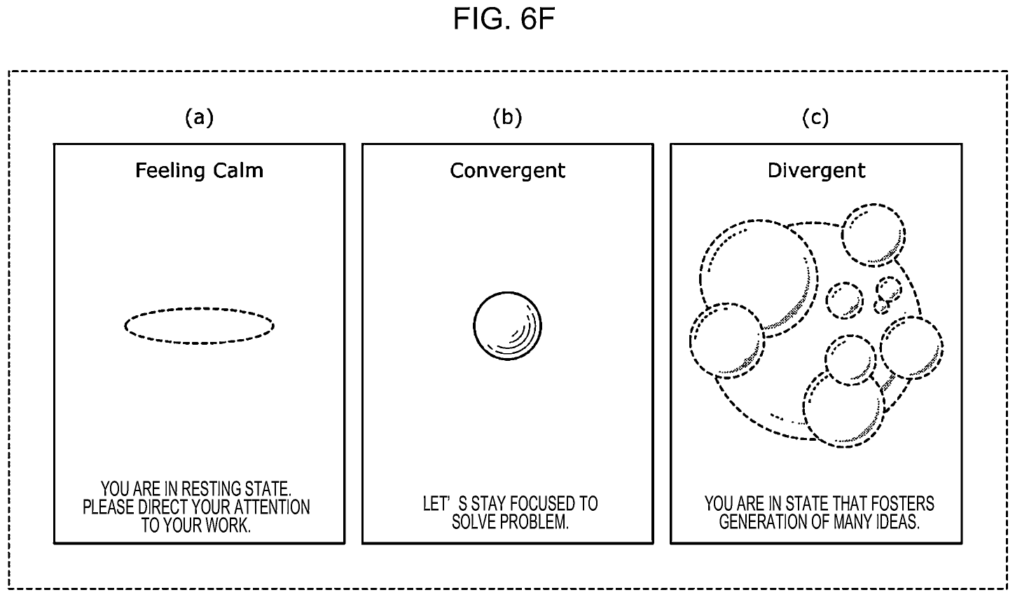

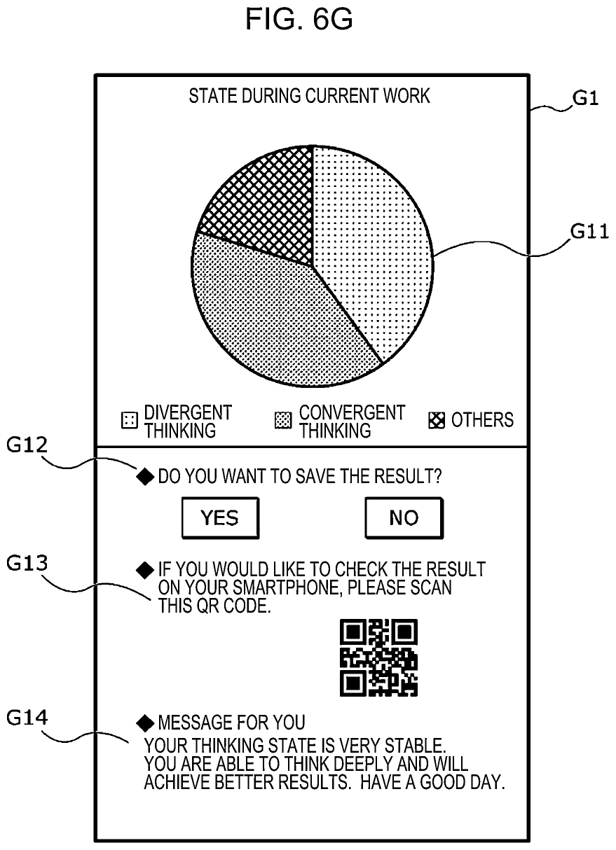

The diagram (for example, a graph) illustrating the determination result presented by the display device 20 may present the thinking state of the user 100 (namely, the convergent thinking state or the divergent thinking state) in time series (FIG. 6D) or may present percentages or ratios of the thinking states of the user 100 (FIG. 6C or 6G). The diagram may represent the thinking state or states of the user 100 only (FIG. 6A, 6C, or 6D) or may represent the thinking state of the user 100 together with its determined evaluation result (FIG. 6B or 6E). Each evaluation value may be a value that indicates the level of validity as the thinking state of the user 100, a value that indicates the level of stability in thinking based on the time period during which a certain thinking state has been maintained continuously, or a value that indicates the percentage of the time period of a certain thinking state in the duration of the task. In a case where the evaluation value is displayed, the evaluation value may be displayed as a score, with the convergent thinking state receiving x points or the divergent thinking state y points (FIG. 6B). The manner in which the evaluation value is displayed may be in the form of an abstract illustration of the thinking state (FIG. 6F). More general expressions may be used to describe the convergent thinking state as “focused” and the divergent thinking state as “creative”. The determination result may be an evaluation determination result of the entire business hours or may be a determination result of a state in a specific time period.

In a case where the determination unit 14 determines the thinking state of the user 100 and thereafter converts the determined thinking state into a score, the score may be determined on the basis of the amount of change from the biometric information of the user 100 acquired in advance in the resting state to the biometric information obtained in the execution state. The score may be calculated by comparing the mean value of the biometric information measured in the execution state of the user 100 with the mean feature value of the data of persons acquired in prior experiments. Furthermore, the score may be calculated by comparing the calculated amount of change from the biometric information of the user 100 acquired in advance in the resting state to the biometric information obtained in the execution state with the mean value of the amounts of variation in the feature data of persons acquired in prior experiments.

Examples of the display of the determination results according to the present embodiment will be illustrated in FIGS. 6A to 6G.

FIG. 6A is a diagram for describing a first example of the display of a determination result according to the present embodiment. FIG. 6A is an example in which the trend of the thinking state of the user 100 during work is presented using words. In FIG. 6A, “divergent thinking” is an abbreviation for “divergent thinking state”. The abbreviation for “convergent thinking state” can be “convergent thinking”. The same will apply thereafter. Note that the display in FIG. 6A is not limited to display on the screen, but may also be presented via audio.

FIG. 6B is a diagram for describing a second example of the display of a determination result according to the present embodiment. FIG. 6B is an example in which the determination result of the thinking state of the user 100 during work is presented in the form of a score. The score displayed may be only a score for a specific thinking state, or the scores for thinking states may be displayed. The maximum score may be 100 points or may be 5 points, for example.

FIG. 6C is a diagram for describing a third example of the display of a determination result according to the present embodiment. FIG. 6C illustrates an example of a pie chart illustrating the percentages of the thinking states of the user 100 (namely, the convergent thinking state or the divergent thinking state) during work.

FIG. 6D is a diagram for describing a fourth example of the display of a determination result according to the present embodiment. FIG. 6D is an example in which the thinking state of the user 100 during work is illustrated in time series. The horizontal axis represents time, and the vertical axis represents the thinking state of the user 100 (namely, the convergent thinking state or the divergent thinking state). Note that the time indicated by the horizontal axis may indicate a single point in time or may indicate several minutes of time.

FIG. 6E is a diagram for describing a fifth example of the display of a determination result according to the present embodiment. FIG. 6E is an example of divergent thinking state scores of the user 100 (also referred to as divergent thinking level) during work in time series. The horizontal axis represents time, and the vertical axis represents divergent thinking state score for the user 100. Note that the vertical axis may represent convergent thinking state score. The results of thinking states may also be displayed in a collective manner.

FIG. 6F includes diagrams for describing a sixth example of the display of determination results according to the present embodiment. FIG. 6F is an example in which the thinking states of the user 100 during work are illustrated as abstract illustrations. For example, a resting state, such as when the user 100 starts working, is displayed as an ellipse with distant foci (see (a) of FIG. 6F).

When the thinking state of the user 100 is determined by the biometric measurement device 1 to be the convergent thinking state, the convergent thinking state is displayed as a circle or sphere with a single focus (see (b) of FIG. 6F). It can be said that this is an abstract illustration of the state in which thoughts come together or reach a single solution through logical thinking.

The dimensions of the circle displayed may be increased as the degree of the divergent thinking state of the user 100 increases. The expansion of thought is expressed as an increase in the dimensions of the circle.

Furthermore, when the thinking state of the user 100 is determined by the biometric measurement device 1 to have a higher divergent thinking level (in other words, the degree of the divergent thinking state is determined to increase) or when the thinking state of the user 100 is determined to be the divergent thinking state, circles that partially overlap are displayed as the divergent thinking state (see (c) of FIG. 6F). It can be said that this is an abstract illustration of the state in which bubbles are arising and diverging or the state in which many ideas are generated through abstract thinking and are coming to mind. The thinking state may be indicated by the tone or saturation of color. For example, an illustration of the convergent thinking state is displayed in a cold color, or an illustration of the divergent thinking state is displayed in a warm color. Illustration shapes or colors are not limited to those described above.

FIG. 6G is a diagram for describing a seventh example of the display of a determination result according to the present embodiment. FIG. 6G is a diagram illustrating an example of presentation including information other than a determination result. FIG. 6G is a diagram illustrating an example of a display image G1 displayed on the display device 20 as presentation information. The display image G1 includes a graph G11, an input field G12, and message display areas G13 and G14.

The graph G11 is substantially the same as the graph illustrated in FIG. 6C.

The input field G12 is a field where the user selects and inputs whether or not to save the determination result. The input field G12 displays the message “Do you want to save the result?”, a “YES” button, and a “NO” button. The “YES” button is for the user to select to save the determination result. The “NO” button is for the user to select not to save the determination result. When the user operates to select the “YES” button, the display device 20 accepts the operation and changes the color of the “YES” button. When the user operates to select the “NO” button, the display device 20 accepts the operation and changes the color of the “NO” button. This allows the user to easily confirm which button has been selected.

When the user operates to select the “YES” button, the display device 20 accepts the operation, saving the determination result is “permitted”, and the determination result regarding the thinking state of the user 100 is saved. When the user operates to select the “NO” button, the display device 20 accepts the operation, saving the determination result is “not permitted”, and the determination result regarding the thinking state of the user 100 is not saved.

The message display area G13 includes a QR code®. The message display area G13 also includes a message to notify the user that the user can check the result from this instance on their mobile terminal by separately scanning the QR code. In FIG. 6G, the message display area G13 includes the message “If you would like to check the result on your smartphone, please scan this QR code” and the QR code. Although the example illustrates a QR code, the user's e-mail address may be entered. Moreover, a card reader may be attached to the display device 20, and in a case where a card, such as an employee ID card, is read by the card reader, the determination result may be transmitted to the user 100 and their supervisor, for example.

The message display area G14 includes a message that takes into account the biometric information of the user 100 during work and the climate and time of day. In FIG. 6G, assuming a user 100 who has been in a specific thinking state for a long time in the early hours of the morning, the positive message “Your thinking state is very stable. You are able to think deeply and will achieve better results. Have a good day.” is displayed.

In the present embodiment, the biometric measurement device 1 may control a lighting device such that the color temperature or illuminance of illuminating light illuminating a surrounding area of the user 100 is changed in accordance with the determination result. For example, the desire of the user 100 to perform a task in a certain thinking state is input to the biometric measurement device 1 in advance. In a case where the task is started in a certain lighting state, the illuminance or color temperature of the room may be changed during the task at regular intervals in accordance with the determination result of the thinking state of the user 100. Moreover, in a case where the user 100 feels comfortable with certain lighting at a particular time, the biometric measurement device 1 may be set to store that lighting setting from an operation terminal connected thereto. The next time the user 100 wants to be in the same thinking state, the user 100 may be able to start the task with that stored lighting setting.

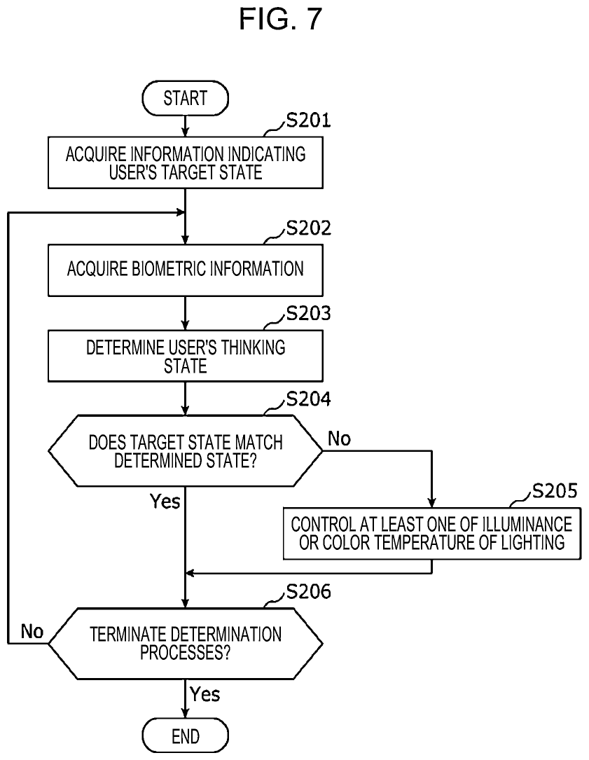

FIG. 7 is a flowchart illustrating an example of a process for controlling lighting in accordance with a determination result according to the present embodiment.

In Step S201, the acquisition unit 11 acquires information regarding the target thinking state of the user 100.

The processes in Steps S202 to S203 are substantially the same as the processes in Steps S101 to S107 in FIG. 3.

In Step S204, the determination unit 14 determines whether or not the target thinking state acquired by the acquisition unit 11 in Step S201 matches the thinking state determined in Step S203. In a case where it is determined that these states match (Yes in Step S204), the process proceeds to Step S206. In a case where it is determined that these states do not match (No in Step S204), the process proceeds to Step S205.

In Step S205, the output unit 15 controls at least one of the illuminance or color temperature of the lighting. Details of the control will be described below.

In Step S206, the determination unit 14 determines whether or not to terminate the series of determination processes illustrated in FIG. 7. For example, the determination unit 14 determines whether or not the acquisition of new biometric information (Step S202) is successful. In a case where it is determined that the acquisition of new biometric information is no longer successful, the determination unit 14 may determine that the series of determination processes illustrated in FIG. 7 is to be terminated. Moreover, the determination unit 14 determines whether or not a preset time has elapsed. In a case where it is determined that the above time has elapsed, the determination unit 14 may determine that the series of determination processes illustrated in FIG. 7 is to be terminated. By terminating the determination processes, the amount of processing of the processor can be reduced.

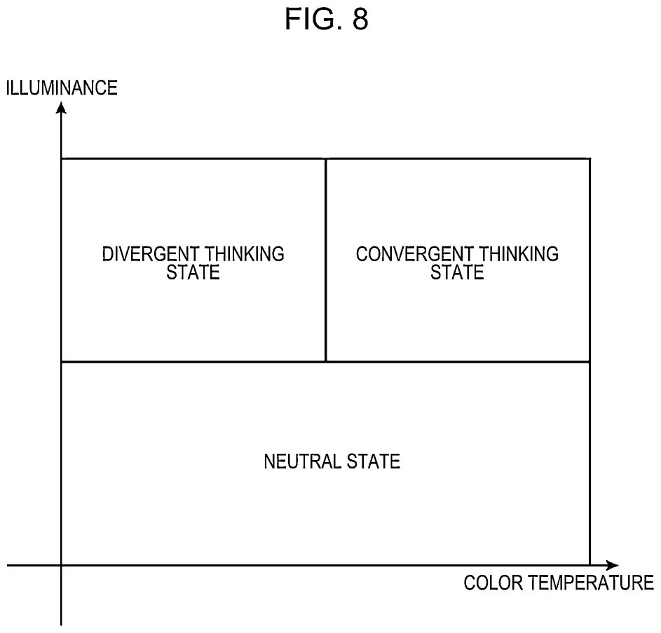

FIG. 8 is a diagram illustrating the relationship between the illuminance and color temperature of lighting suitable for inducing the user 100 to a certain thinking state (namely, the convergent thinking state or the divergent thinking state). The relationship between the illuminance and color temperature of such lighting is described in the work by Koichiro FUMOTO, Yutaka HASHIURA, Kouzou TSUJI, and Takeki KIMURA titled “Office lighting system to encourage creativity of workers—Study on optimal lighting conditions for creative work in the office—,” presented in Japan Human Factors and Ergonomics Society Kansai Branch Rombun-shu 2009, pages 171-174, Dec. 5, 2009.

The biometric measurement device 1 can thus induce the user 100 to the divergent thinking state or the convergent thinking state by controlling the lighting device, as one example.

### (1) Control to Induce User 100 to Divergent Thinking State

For example, the use of lighting with high illuminance and low color temperature as conditions suitable for work such as brainstorming, namely, conditions suitable for the divergent thinking state is described in the work by Koichiro FUMOTO, Yutaka HASHIURA, Kouzou TSUJI, and Takeki KIMURA titled “Office lighting system to encourage creativity of workers—Study on optimal lighting conditions for creative work in the office—,” presented in Japan Human Factors and Ergonomics Society Kansai Branch Rombun-shu 2009, pages 171-174, Dec. 5, 2009.

In contrast, when it is desired to induce the user 100 to the divergent thinking state, the thinking state of the user 100 is presumed to be different from the divergent thinking state and to be the convergent thinking state or a neutral state, for example.

In this case, in a case where the biometric measurement device 1 controls the lighting device to lower the color temperature of the illuminating light and increase the illuminance of the illuminating light, it may be possible to induce the user 100 to the divergent thinking state (see FIG. 8).

Thus, in a case where at least one index out of the amount of variation in the face position of the user 100, the amount of variation in the face orientation of the user 100, the amount of variation in the gaze direction of the user 100, and the eye closure percentage of the user 100 is determined to be less than the first threshold and where the at least one index described above is determined to be greater than or equal to the second threshold (that is, the user 100 is determined to be in the convergent thinking state), when the biometric measurement device 1 controls the lighting device to lower the color temperature of the illuminating light and increase the illuminance of the illuminating light, it may be possible to induce the user 100 to the divergent thinking state.

Moreover, in a case where at least one index out of the amount of variation in the face position of the user 100, the amount of variation in the face orientation of the user 100, the amount of variation in the gaze direction of the user 100, and the eye closure percentage of the user 100 is determined to be less than the second threshold (that is, the user 100 is determined to be in the neutral state), when the biometric measurement device 1 controls the lighting device to lower the color temperature of the illuminating light and increase the illuminance of the illuminating light, it may be possible to induce the user 100 to the divergent thinking state.

### (2) Control to Induce User 100 to Convergent Thinking State

For example, the use of lighting with high color temperature and high illuminance as conditions suitable for the convergent thinking state is described in the work by Koichiro FUMOTO, Yutaka HASHIURA, Kouzou TSUJI, and Takeki KIMURA titled “Office lighting system to encourage creativity of workers—Study on optimal lighting conditions for creative work in the office—,” presented in Japan Human Factors and Ergonomics Society Kansai Branch Rombun-shu 2009, pages 171-174, Dec. 5, 2009.

In contrast, when it is desired to induce the user 100 to the convergent thinking state, the thinking state of the user 100 is presumed to be different from the convergent thinking state and to be the divergent thinking state or the neutral state, for example.

In this case, in a case where the biometric measurement device 1 controls the lighting device to increase the color temperature of the illuminating light and increase the illuminance of the illuminating light, it may be possible to induce the user 100 to the convergent thinking state (see FIG. 8).

Thus, in a case where at least one index out of the amount of variation in the face position of the user 100, the amount of variation in the face orientation of the user 100, the amount of variation in the gaze direction of the user 100, and the eye closure percentage of the user 100 is determined to be greater than or equal to the first threshold (that is, the user is determined to be in the divergent thinking state), when the biometric measurement device 1 controls the lighting device to increase the color temperature of the illuminating light and increase the illuminance of the illuminating light, it may be possible to induce the user 100 to the convergent thinking state (see FIG. 8).

Moreover, in a case where at least one index out of the amount of variation in the face position of the user 100, the amount of variation in the face orientation of the user 100, the amount of variation in the gaze direction of the user 100, and the eye closure percentage of the user 100 is determined to be less than the second threshold (that is, the user is determined to be in the neutral state), when the biometric measurement device 1 controls the lighting device to increase the color temperature of the illuminating light and increase the illuminance of the illuminating light, it may be possible to induce the user 100 to the convergent thinking state (see FIG. 8).

The biometric measurement device 1 can also determine the thinking state depending on the type of task being engaged in. In this case, the biometric measurement device 1 inputs information regarding the task statuses of the user 100 working on tasks, calculates a mean value from the biometric information obtained in the time region required for each task, and determines the thinking state of the user 100 while the user 100 was engaged in each task.

In the determination process as illustrated in FIG. 4, for example, threshold information for determining the value to which the threshold is to be set is stored in the memory device, and a threshold set using the threshold information acquired from the memory device may be used.

The threshold information may be stored in association with an identification (ID) for identifying an individual, the user 100. In this case, the threshold appropriate for the individual, the user 100, can be used to determine their thinking state, thereby improving the accuracy of the determination.

The ID-associated threshold information may be a value that takes into account the amount of variation in the face position of the user 100, the amount of variation in the face orientation of the user 100, the amount of variation in the gaze direction of the user 100, and the eye closure percentage of the user 100 being in their resting state. Regarding users 100, the amounts of variation in face position, the amounts of variation in face orientation, the amounts of variation in gaze direction, and the eye closure percentages for the individual users 100 being in their resting state may be stored in association with their IDs in the memory device, and the threshold information for each individual user 100 may be generated. The resting state may be a neutral state (in other words, a neutral state in terms of thinking) induced by having the users 100 listen to white noise or utter meaningless words. Alternatively, the resting state may be a state in which the users 100 are not engaged in a task or other activity. The threshold information may be updated on the basis of thinking state determination results.

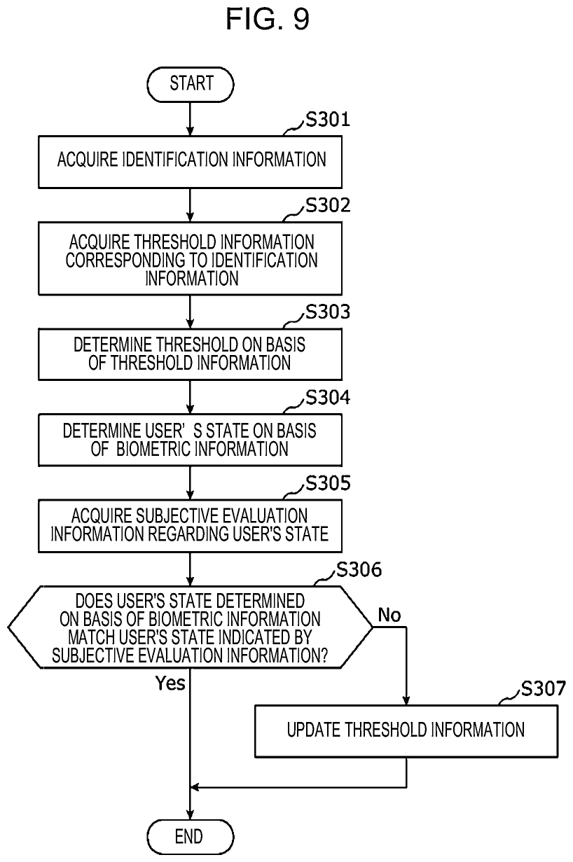

FIG. 9 is a flowchart illustrating an example of a process regarding the update of threshold information according to the present embodiment. FIG. 9 illustrates a process for making a determination using the threshold information associated with the identification information of a user 100.

In Step S301, the determination unit 14 acquires the identification information of the user 100. The identification information is information that can uniquely identify the user 100. The identification information may be letters or symbols entered by the user 100 using input devices, such as a keyboard, or may be an image of the face of the user 100 captured by the camera 10.

In Step S302, the determination unit 14 acquires, from the memory device, the threshold information corresponding to the identification information acquired in Step S301.

In Step S303, the determination unit 14 determines a threshold on the basis of the threshold information acquired in Step S302. The threshold determined is a first threshold corresponding to at least one index out of the amount of variation in face position, the amount of variation in face orientation, the amount of variation in gaze direction, and the eye closure percentage. Note that the threshold determined may be a second threshold corresponding to at least one index out of the amount of variation in face position, the amount of variation in face orientation, the amount of variation in gaze direction, and the eye closure percentage.

In Step S304, the determination unit 14 performs a determination process based on the biometric information of the user 100. The determination process performed in Step S304 corresponds to the process illustrated in FIG. 4.

In Step S305, the determination unit 14 acquires subjective evaluation information regarding the state of the user 100. The subjective evaluation information corresponds to the thinking state of the user 100 evaluated by the user 100 themselves. The determination unit 14 causes a graphical user interface (GUI) to be presented. The GUI includes, for example, a message asking about the thinking state of the user 100 during the task, such as “Please self-evaluate your state during the task.”, and options for the state of the user 100 during the task (specifically, “divergent thinking state” and “convergent thinking state”) and accepts a response from the user. In a case where the user 100 responds with “divergent thinking state” using the GUI, the information representing that the user 100 was in the divergent thinking state while performing the task is acquired as the subjective evaluation information. The information acquired in Step S305 is not limited to the subjective evaluation information obtained as a result of evaluation performed by the examinee themselves, and a determination result determined using a method different from that used to determine the thinking state based on heart rate may also be acquired as evaluation information.

In Step S306, the determination unit 14 determines whether or not the state of the user 100 determined in Step S304 matches the state of the user 100 indicated by the subjective evaluation information acquired in Step S305. For example, in a case where the state of the user 100 determined on the basis of the biometric information is the convergent thinking state and where the state of the user 100 indicated by the subjective evaluation information is the convergent thinking state, the determination unit 14 determines that these states match. In contrast, for example, in a case where the state of the user 100 determined on the basis of the biometric information is the convergent thinking state and where the state of the user 100 indicated by the subjective evaluation information is the divergent thinking state, the determination unit 14 determines that these states do not match.

In a case where these states match (Yes in Step S306), the series of processes illustrated in FIG. 9 is terminated. Otherwise (No in Step S306), the process proceeds to Step S307.

In Step S307, the determination unit 14 updates the threshold information associated with the user 100 (namely, the identification information) on the basis of the determined state of the user 100 and the state of the user 100 indicated by the subjective evaluation information. For example, in a case where the state of the user 100 determined on the basis of the biometric information is the convergent thinking state and where the state of the user 100 indicated by the subjective evaluation information is the divergent thinking state, the threshold information is updated so as to lower the first threshold for the amount of variation in face position indicated by the threshold information. This is because lowering the first threshold for the amount of variation in face position can expand the range of the amount of variation in face position that is determined to be greater than or equal to the first threshold (Step S102) and contribute to determining the state of the user 100 to be the divergent thinking state.

As described above, the biometric measurement device 1 can improve the accuracy of thinking state determinations by updating the threshold information using the results of subjective evaluations.

In the embodiment described above, each structural element may be configured using dedicated hardware or may be realized by executing a software program suitable for each structural element. Each structural element may be realized by a program execution unit, such as a central processing unit (CPU) or processor, reading out and executing a software program recorded on a recording medium, such as a hard disk or semiconductor memory. In this case, the software that realizes the information processing device, for example, according to the embodiment described above is a program as follows.

That is, this program causes the computer to perform, using a processor, an information processing method that includes acquiring at least one index selected from an index group including the amount of variation in a face position of a user, the amount of variation in a face orientation of the user, the amount of variation in a gaze direction of the user, and an eye closure percentage of the user, and determining whether or not the user is in the divergent thinking state on the basis of the acquired at least one index.

The configuration may also be such that any of the CPU and processor, which perform the thinking state determination process and that the memory device is provided in a server that communicates via a network with a sensor that detects the user's biometric information.

As described above, for example, the information processing methods according to one or more aspects have been described on the basis of the embodiment; however, the present disclosure is not limited to this embodiment. Forms obtained by adding various modifications that one skilled in the art can conceive of to the present embodiment, as well as forms constructed by combining structural elements in different embodiments, may also be included in the scope of the one or more aspects, as long as these forms do not depart from the gist of the present disclosure.

### Others

Modifications of the embodiment of the present disclosure may be those described below.

### Item 1

A method performed by a processor, the method including
acquiring one or more values included in a value V_{FP} of a first index, a value V_{FD} of a second index, a value V_{ED} of a third index, and a value V_{EC} of a fourth index,
the first index indicating a variation in a face position of a user, the second index indicating a variation in a face orientation of the user, the third index indicating a variation in a gaze direction of the user, the fourth index indicating a percentage of time the user keeps both eyes closed, and
determining, based on the acquired one or more values, whether or not the user is in a divergent thinking state.

### Item 2

The method according to the item 1, further including
acquiring images I1(1) to I1(n), the images I1(1) to I1(n) being images of the face of the user captured at first predetermined intervals in a first period in which the user is not performing a task, the image I1(i) being captured at a time t1i, i=1 to n,
determining, based on the image I1(i), a value fp1(i) indicating a face position of the user at the time t1i, a value fd1(i) indicating a face orientation of the user at the time t1i, a value ed1(i) indicating a gaze direction of the user at the time t1i, and a value ec1(i) indicating whether or not the eyes of the user are closed at the time t1i, thereby fp1(1) to fp1(n), fd1(1) to fd1(n), ed1(1) to ed1(n), and ec1(1) to ec1(n) being determined, ec1(i) being equal to 1 in a case where the eyes of the user are closed at the time t1i, ec1(i) being equal to 0 in a case where the eyes of the user are not closed at the time t1i,
determining FP1, based on the fp1(1) to the fp1(n),
determining FD1, based on the fd1(1) to the fd1(n),
determining ED1, based on the ed1(1) to the ed1(n),
determining EC1, based on the ec1(1) to the ec1(n),
acquiring images I2(1) to I2(m), the images 12(1) to 12(m) being images of the face of the user captured at second predetermined intervals in a second period in which the user is performing a task, the image 12(j) being captured at a time t2j, j=1 to m,
determining, based on the image I2(j), a value fp2(j) indicating a face position of the user at the time t2j, a value fd2(j) indicating a face orientation of the user at the time t2j, a value ed2(j) indicating a gaze direction of the user at the time t2j, and a value ec2(j) indicating whether or not the eyes of the user are closed at the time t2j, thereby fp2(1) to fp2(m), fd2(1) to fd2(m), ed2(1) to ed2(m), and ec2(1) to ec2(m) being determined, ec2(j) being equal to 1 in a case where the eyes of the user are closed at the time t2j, ec2(j) being equal to 0 in a case where the eyes of the user are not closed at the time t2j,
determining FP2, based on the fp2(1) to the fp2(m),
determining FD2, based on the fd2(1) to the fd2(m),
determining ED2, based on the ed2(1) to the ed2(m), and
determining EC2, based on the ec2(1) to the ec2(m), in which

$${V}_{FP} = ( FP 2 - FP 1 ) ,$$

$${V}_{FD} = ( FD 2 - FD 1 ) ,$$

$${V}_{ED} = ( ED 2 - ED 1 ) , and$$

$${V}_{EC} = ( EC 2 - EC 1 ) .$$

The first period may be smaller than the second period, and n may be smaller than m.

The amount of processing performed by the processor in a case where n<m is less than the amount of processing performed by the processor in a case where n=m. The memory consumption in a case where processing is performed under the condition that n<m is smaller than the memory consumption in a case where processing is performed under the condition that n=m.

The time required to determine the variation in the value indicating the face position of the user, the variation in the value indicating the face orientation of the user, the variation in the value indicating the gaze direction of the user, and the variation in the value indicating whether or not the eyes of the user are closed in the first period during which the user is not performing a task is
sufficient even if the time is shorter than the time required to determine the variation in the value indicating the face position of the user, the variation in the value indicating the face orientation of the user, the variation in the value indicating the gaze direction of the user, and the variation in the value indicating whether or not the eyes of the user are closed in the second period during which the user is performing a task.

The first period may be one minute, the n may be the number of images captured during the first period, each of the first predetermined intervals may be 0.1 seconds, and the n may be 600. Each of the first predetermined intervals may be greater than or equal to (1/20) seconds and less than or equal to (1/10) seconds.

The second period may be three minutes, the m may be the number of images captured during the second period, each of the second predetermined intervals may be 0.1 seconds, and the m may be 1800. Each of the second predetermined intervals may be greater than or equal to (1/20) seconds and less than or equal to (1/10) seconds.

### Item 3

The method according to the item 2, in which
the FP1 is a standard deviation of the fp1(1), . . . , the fp1(n),
the FD1 is a standard deviation of the fd1(1), . . . , the fd1(n),
the ED1 is a standard deviation of the ed1(1), . . . , the ed1(n),
the EC1 is (ec1(1)+ . . . +ec1(n))/n,
the FP2 is a standard deviation of the fp2(1), . . . , the fp2(m),
the FD2 is a standard deviation of the fd2(1), . . . , the fd2(m),
the ED2 is a standard deviation of the ed2(1), . . . , the ed2(m), and
the EC2 is (ec2(1)+ . . . +ec2(m))/m.

### Item 4

The method according to the item 2, in which
the FP1 is a variance of the fp1(1), . . . , the fp1(n),
the FD1 is a variance of the fd1(1), . . . , the fd1(n),
the ED1 is a variance of the ed1(1), . . . , the ed1(n),
the EC1 is (ec1(1)+ . . . +ec1(n))/n,
the FP2 is a variance of the fp2(1), . . . , the fp2(m),
the FD2 is a variance of the fd2(1), . . . , the fd2(m),
the ED2 is a variance of the ed2(1), . . . , the ed2(m), and
the EC2 is (ec2(1)+ . . . +ec2(m))/m.

### Item 5

The method according to the item 2, in which
first thresholds include a threshold (11) corresponding to the first index, a threshold (12) corresponding to the second index, a threshold (13) corresponding to the third index, and a threshold (14) corresponding to the fourth index,
the acquired one or more values includes a value corresponding to the j-th index,
the processor determines in a case where the value is greater than or equal to the threshold (1j) that the user is in a divergent thinking state, and

$$j = 1 , j = 2 , j = 3 , or j = 4.$$

### Item 6

The method according to the item 2, in which
first thresholds include a threshold (11) corresponding to the first index, a threshold (12) corresponding to the second index, a threshold (13) corresponding to the third index, and a threshold (14) corresponding to the fourth index,
second thresholds include a threshold (21) corresponding to the first index, a threshold (22) corresponding to the second index, a threshold (23) corresponding to the third index, and a threshold (24) corresponding to the fourth index,
each of the acquired one or more values is smaller than a threshold that is included in the first thresholds and corresponds to each of the acquired one or more values,
a value included in the acquired one or more values is a value corresponding to the k-th index,
in a case where the value is greater than or equal to the threshold (2k), the processor determines that the user is in a convergent thinking state,

$$k = 1 , k = 2 , k = 3 , or k = 4 ,$$

the threshold (11) is greater than the threshold (21),
the threshold (12) is greater than the threshold (22),
the threshold (13) is greater than the threshold (23), and
the threshold (14) is greater than the threshold (24).

“Each of the acquired one or more values is smaller than a threshold that is included in the first thresholds and corresponds to each of the acquired one or more values” may be interpreted as in (a) to (d) below.

(a) In a case where the acquired one or more values are a first value, (the first value)<(a threshold corresponding to the first value).

The first value is FP1, FD1, ED1, or EC1.

In a case where the first value is FP1, (the threshold corresponding to the first value) is the threshold (11).

In a case where the first value is FD1, (the threshold corresponding to the first value) is the threshold (12).

In a case where the first value is ED1, (the threshold corresponding to the first value) is the threshold (13).

In a case where the first value is EC1, (the threshold corresponding to the first value) is the threshold (14).

(b) In a case where the acquired one or more values correspond to the first value and a second value, (the first value)<(the threshold corresponding to the first value), and (the second value)<(a threshold corresponding to the second value).

The first value is FP1, FD1, ED1, or EC1. The second value is FP1, FD1, ED1, or EC1. Note that the first value and the second value are different.

In a case where the first value is FP1, (the threshold corresponding to the first value) is the threshold (11).

In a case where the first value is FD1, (the threshold corresponding to the first value) is the threshold (12).

In a case where the first value is ED1, (the threshold corresponding to the first value) is the threshold (13).

In a case where the first value is EC1, (the threshold corresponding to the first value) is the threshold (14).

In a case where the second value is FP1, (the threshold corresponding to the second value) is the threshold (11).

In a case where the second value is FD1, (the threshold corresponding to the second value) is the threshold (12).

In a case where the second value is ED1, (the threshold corresponding to the second value) is the threshold (13).

In a case where the second value is EC1, (the threshold corresponding to the second value) is the threshold (14).

(c) In a case where the acquired one or more values correspond to the first value, the second value, and a third value, (the first value)<(the threshold corresponding to the first value), (the second value)<(the threshold corresponding to the second value), and (the third value)<(a threshold corresponding to the third value).

The first value is FP1, FD1, ED1, or EC1. The second value is FP1, FD1, ED1, or EC1. The third value is FP1, FD1, ED1, or EC1. Note that the first value, the second value, and the third value are different from each other.

In a case where the first value is FP1, (the threshold corresponding to the first value) is the threshold (11).

In a case where the first value is FD1, (the threshold corresponding to the first value) is the threshold (12).

In a case where the first value is ED1, (the threshold corresponding to the first value) is the threshold (13).

In a case where the first value is EC1, (the threshold corresponding to the first value) is the threshold (14).

In a case where the second value is FP1, (the threshold corresponding to the second value) is the threshold (11).

In a case where the second value is FD1, (the threshold corresponding to the second value) is the threshold (12).

In a case where the second value is ED1, (the threshold corresponding to the second value) is the threshold (13).

In a case where the second value is EC1, (the threshold corresponding to the second value) is the threshold (14).

In a case where the third value is FP1, (the threshold corresponding to the third value) is the threshold (11).

In a case where the third value is FD1, (the threshold corresponding to the third value) is the threshold (12).

In a case where the third value is ED1, (the threshold corresponding to the third value) is the threshold (13).

In a case where the third value is EC1, (the threshold corresponding to the third value) is the threshold (14).

(d) In a case where the acquired one or more values correspond to FP1, FD1, ED1, and EC1,
FP1<the threshold (11), FD1<the threshold (12), ED1<the threshold (13), and EC1<the threshold (14).

The present disclosure is applicable to devices that sense people's states.

What is claimed is:

1. An information processing method comprising:
by using a processor,
acquiring at least one index selected from an index group that includes an amount of variation in a face position of a user, an amount of variation in a face orientation of the user, an amount of variation in a gaze direction of the user, and an eye closure percentage of the user; and
determining, based on the at least one acquired index, whether or not the user is in a divergent thinking state.

2. The information processing method according to claim 1, wherein
the determining includes
comparing the at least one index with a first threshold corresponding to the at least one index, and
determining, in a case where the at least one index is determined to be greater than or equal to the first threshold, that the user is in the divergent thinking state.

3. The information processing method according to claim 2, wherein
the determining includes
comparing the at least one index with a second threshold corresponding to the at least one index and different from the first threshold, and
determining, in a case where the at least one index is determined to be less than the first threshold and greater than or equal to the second threshold, that the user is in a convergent thinking state.

4. The information processing method according to claim 2, wherein
the determining includes
determining, for each of indices selected from the index group, whether or not the index is greater than or equal to the first threshold corresponding thereto.

5. The information processing method according to claim 4, wherein
the determining includes
determining, in a case where the amount of variation in the face orientation of the user is determined to be greater than or equal to the first threshold corresponding thereto and where the amount of variation in the gaze direction of the user is determined to be greater than or equal to the first threshold corresponding thereto, that the user is in the divergent thinking state.

6. The information processing method according to claim 4, wherein
the determining includes
determining a degree of the divergent thinking state of the user in a unit period, based on a count of indices that are greater than or equal to the first thresholds corresponding thereto among the indices acquired in the unit period.

7. The information processing method according to claim 3, wherein
the determining includes
determining, using the at least one index acquired in each of unit periods, whether or not the user is in the divergent thinking state and whether or not the user is in the convergent thinking state in each of the unit periods,
generating a graph representing a percentage of a period during which the user is determined to be in the convergent thinking state and a percentage of a period during which the user is determined to be in the divergent thinking state, and
displaying the graph.

8. The information processing method according to claim 2, further comprising:
acquiring identification information for identifying the user; and
acquiring threshold information corresponding to the identification information,
wherein the first threshold is determined based on the threshold information.

9. The information processing method according to claim 8, further comprising:
acquiring evaluation information obtained by evaluating a thinking state of the user using a method different from the determination process; and
updating the threshold information based on a determination result of a determined thinking state of the user and the evaluation information.

10. The information processing method according to claim 3, further comprising:
controlling, in a case where the at least one index is determined to be less than the first threshold and greater than or equal to the second threshold, a lighting device, which illuminates a surrounding area of the user, to lower a color temperature of illuminating light and increase illuminance of the illuminating light.

11. The information processing method according to claim 2, further comprising:
controlling, in a case where the at least one index is determined to be greater than or equal to the first threshold, a lighting device, which illuminates a surrounding area of the user, to increase a color temperature of illuminating light and increase illuminance of the illuminating light.

12. The information processing method according to claim 3, further comprising:
controlling, in a case where the at least one index is determined to be less than the second threshold, a lighting device, which illuminates a surrounding area of the user, to
(i) lower a color temperature of illuminating light and increase illuminance of the illuminating light, or
(ii) increase a color temperature of illuminating light and increase illuminance of the illuminating light.

13. The information processing method according to claim 1, wherein
the acquiring includes
acquiring an image generated by an image capturing device that captures the user, and
acquiring the at least one index by performing an analysis process on the acquired image.

14. An information processing method comprising:
by using a processor,
acquiring target information indicating a target state that is a state targeted by a user;
acquiring at least one index selected from an index group that includes an amount of variation in a face position of the user, an amount of variation in a face orientation of the user, an amount of variation in a gaze direction of the user, and an eye closure percentage of the user;
determining a thinking state of the user based on the at least one index;
comparing the target state indicated by the target information with the determined thinking state of the user; and
performing, in a case where the target state and the determined thinking state of the user are different as a result of the comparison, control to change a characteristic of illuminating light emitted from a lighting device, which illuminates a surrounding area of the user.

15. An information processing system comprising:
an image capturing device that captures a user to generate an image;
a processing circuit;
a memory device; and
a display device, wherein
the processing circuit
acquires at least one index selected from an index group that includes an amount of variation in a face position of the user, an amount of variation in a face orientation of the user, an amount of variation in a gaze direction of the user, and an eye closure percentage of the user in a unit period by performing an analysis process on the image generated by the image capturing device,
acquires, from the memory device, threshold information indicating a first threshold corresponding to the at least one index,
performs a determination process for determining whether or not the user is in a divergent thinking state in the unit period, based on a determining result as to whether or not the at least one index is greater than or equal to the first threshold in the unit period, and
causes the display device to display an image corresponding to a result of the determination process.
